SEQUENCE LISTING (4 sequences)

SEQ ID NO: 1 - DNA, 954 bp, Homo sapiens
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 2 - DNA, 20 bp, Homo sapiens
ctgatggacgagaccatgaa

SEQ ID NO: 3 - DNA, 20 bp, Homo sapiens
cggctgggcgcggacatgga

SEQ ID NO: 4 - DNA, 20 bp, Homo sapiens
caggccggggcccgcgaggg

(19) United States
(12) Patent Application Publication
(10) Pub. No.: US 2009/0136456 A1
(43) Pub. Date: May 28, 2009

(54) METHODS OF TREATING NEURODEGENERATIVE DISORDERS

(76) Inventors: Yadong Huang, San Francisco, CA (US); Gang Li, San Francisco, CA (US); Robert W. Mahley, San Francisco, CA (US); Qin Xu, Burlingame, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/961,951

(22) Filed: Dec. 20, 2007

Related U.S. Application Data
(60) Provisional application No. 60/876,944, filed on Dec. 22, 2006.

(51) Int. Cl. A61K 35/12; C12N 5/00; A61P 25/28

(52) U.S. Cl. 424/93.7; 435/325

(57) ABSTRACT

The present invention provides a selected population of neural cells, including neural stem cells, neural progenitor cells, neural precursor cells, and progeny thereof, which neural cells are selected for an apoE4⁻ phenotype. In some embodiments, the neural cells are further selected for an apoE3⁺ phenotype. The selected population of neural cells is useful in treating various disorders, such as neurodegenerative disorders and demyelination diseases. The present invention further provides methods of treating neurodegenerative disorders and demyelinating diseases, generally involving administering a subject selected cell population.

### CROSS-REFERENCE

This application claims the benefit of U.S. Provisional Patent Application No. 60/876,944, filed Dec. 22, 2006, which application is incorporated herein by reference in its entirety.

### BACKGROUND

Apolipoprotein E (apoE), a 34,000 molecular weight protein, is the product of a single gene on chromosome 19 and exists in three major isoforms designated apoE2, apoE3 and apoE4. ApoE mRNA is abundant in the brain, where it is synthesized and secreted primarily by astrocytes. Although apoE is synthesized in the brain primarily by astrocytes, neurons in the central nervous system (CNS) express apoE in response to excitotoxic stress and other insults.

It has been shown that neuronal expression of apoE, especially apoE4, contributes to the pathogenesis of Alzheimer's Disease (AD), such as neurofibrillary tangle formation and mitochondrial dysfunction. ApoE4 is a major risk factor for AD. AD patients with apoE4 have greater hippocampal atrophy than those without apoE4, and even normal middle-aged subjects with apoE4 have a smaller hippocampus, a brain structure responsible for normal learning and memory. ApoE4 is also associated with poor clinical outcome and with earlier onset, progression, or severity of head trauma, stroke, Parkinson's disease, multiple sclerosis, and amyotrophic lateral sclerosis.

There are a number of neurodegenerative disorders for which there is currently no effective treatment. There is a need in the art for effective treatments for various neurodegenerative disorders.

### Literature

U.S. Pat. Nos. 5,980,885, 6,497,872, 6,680,198, 6,713,247, and 6,777,233.

### SUMMARY OF THE INVENTION

The present invention provides a selected population of neural cells, including neural stem cells, neural progenitor cells, neural precursor cells, and progeny thereof, which neural cells are selected for one or both of an apoE4⁻ phenotype and an apoE3⁺ phenotype. The selected population of neural cells is useful in treating various disorders, such as neurodegenerative disorders and demyelination diseases. The present invention further provides methods of treating neurodegenerative disorders and demyelinating diseases, generally involving administering a subject selected cell population.

### BRIEF DESCRIPTION OF THE DRAWINGS

FIG. 1 is a schematic depiction of a construct for generating an enhanced green fluorescent protein (EGFP) knock-in mouse (EGFP_{apoE} reporter mouse), in which the EGFP cDNA was inserted into the mouse apoE locus, by gene targeting, immediately after the translation initiation site.

FIG. 2 depicts expression of EGFP (representing apoE expression) in EGFP-targeted mouse stem cells in response to neuronal differentiation.

FIG. 3 depicts expression of EGFP (representing apoE expression) in response to C6-conditioned medium in neurons differentiated from EGFP-targeted embryonic stem (ES) cells.

FIG. 4 depicts the expression levels of apoE mRNA and protein in heterozygous EGFP knock-in (EGFP_{apoE} reporter) mice.

FIG. 5 depicts expression of EGFP (representing apoE expression) in dentate gyrus of the hippocampus of EGFP knock-in (EGFP_{apoE} reporter) mice.

FIG. 6 depicts expression of EGFP (representing apoE expression) in nestin-positive neuronal progenitors in the hippocampus of EGFP knock-in (EGFP_{apoE} reporter) mice.

FIG. 7 schematically depicts a protocol for measuring adult hippocampal neurogenesis in mice.

FIGS. 8A-D depict the effects of apoE on hippocampal neurogenesis. The number of BrdU-positive cells (newborn cells; immature neurons; mature neurons; and astrocytes) at various time points after BrdU injection, in apoE3-KI mice, apoE4-KI mice, and apoE-knock-out (apoE-KO) mice, is shown.

FIG. 9 depicts isoform-dependent effect of apoE on differentiation and maturation of survived cells, 4 weeks after BrdU injection.

FIG. 10 depicts the number of BrdU-positive cells in the subgranular zone of the hippocampus in apoE3-KI and apoE4-KI mice at different ages, 1 day after BrdU injection.

FIG. 11 depicts the number of BrdU/S100β double positive cells (astrocytes) per hippocampus in apoE3-KI mice, apoE4-KI mice, apoE-KO mice, GFAP-E3 mice, and GFAP-E4 mice, 3 days after BrdU injection.

FIG. 12 depicts the number of BrdU/Dcx double positive cells (immature neurons) per hippocampus, 3 days after BrdU injection, in apoE3-KI mice, apoE4-KI mice, apoE-KO mice, GFAP-E3 mice, and GFAP-E4 mice.

### DEFINITIONS

As used herein, the term “neural stem cell” refers to an undifferentiated neural cell that can be induced to proliferate. A neural stem cell can be derived from an embryonic stem cell, an adult stem cell, or an induced pluripotent stem (iPS) cell. The neural stem cell is capable of self-maintenance, meaning that with each cell division, one daughter cell will also be a stem cell. The non-stem cell progeny of a neural stem cell are termed progenitor cells. The progenitor cells generated from a single multipotent neural stem cell are capable of differentiating into neurons, astrocytes (type I and type II) and oligodendrocytes. Hence, the neural stem cell is “multipotent” because its progeny have multiple differentiative pathways.

The term “induced pluripotent stem cell” (or “iPS cell”), as used herein, refers to a pluripotent stem cell induced from a somatic cell, e.g., a differentiated somatic cell. iPS cells are capable of self-renewal and differentiation into cell fate-committed stem cells, including neural stem cells, as well as various types of mature cells.

The term “neural progenitor cell”, as used herein, refers to an undifferentiated cell derived from a neural stem cell, and is not itself a stem cell. Some progenitor cells can produce progeny that are capable of differentiating into more than one cell type.

The term “precursor cells”, as used herein, refers to the progeny of neural stem cells, and thus includes both progenitor cells and daughter neural stem cells.

The terms “short interfering nucleic acid,” “siNA,” “short interfering RNA,” “siRNA,” “short interfering nucleic acid molecule,” “short interfering oligonucleotide molecule,” or “chemically-modified short interfering nucleic acid molecule,” as used herein, refer to any nucleic acid molecule capable of inhibiting or down regulating gene expression, for example by mediating RNA interference “RNAi” or gene silencing in a sequence-specific manner. Design of RNAi molecules when given a target gene is routine in the art. See also US 2005/0282188 (which is incorporated herein by reference) as well as references cited therein. See, e.g., Pushparaj et al. Clin Exp Pharmacol Physiol. 2006 May-June; 33(5-6):504-10; Lutzelberger et al. Handb Exp Pharmacol. 2006; (173):243-59; Aronin et al. Gene Ther. 2006 March; 13(6):509-16; Xie et al. Drug Discov Today. 2006 January; 11 (1-2):67-73; Grunweller et al. Curr Med. Chem. 2005; 12(26):3143-61; and Pekaraik et al. Brain Res Bull. 2005 Dec. 15; 68(1-2):115-20. Epub 2005 Sep. 9.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” means the amount of a compound or a number of cells that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a neural cell” includes a plurality of such cells and reference to “the cell population” includes reference to one or more cell populations and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides a selected population of neural cells, including neural stem cells, neural progenitor cells, neural precursor cells, and progeny thereof, which neural cells are selected for an apoE4⁻ phenotype. In some embodiments, the neural cells are further selected for an apoE3⁺ phenotype. The selected population of neural cells is useful in treating various disorders, such as neurodegenerative disorders and demyelination diseases. The present invention further provides methods of treating neurodegenerative disorders and demyelinating diseases, generally involving administering a subject selected cell population.

### Selected Neural Cell Population

The present invention provides a selected population of neural cells, including neural stem cells, neural progenitor cells, neural precursor cells, and progeny thereof, which neural cells are selected for an apolipoprotein E4-negative (apoE4⁻) phenotype. In some embodiments, the neural cells are further selected for an apolipoprotein E3-positive (apoE3⁺) phenotype. In some embodiments, the neural cell is genetically modified such that the genetically modified neural cell overexpresses apoE3 and/or such that apoE4 expression, if any, is knocked down (e.g., reduced or eliminated).

The selected population of neural cells is useful in treating various disorders, such as neurodegenerative disorders and demyelination diseases. The present invention further provides compositions, including pharmaceutical compositions, comprising the selected cell population.

Neural stem cells of various species have been described. See, e.g., WO 93/01275, WO 94/09119, WO 94/10292, WO 94/16718, and Cattaneo et al., Mol. Brain. Res., 42, pp. 161-66 (1996). In some embodiments, central nervous system (CNS) neural stem cells, when maintained in a mitogen-containing (typically epidermal growth factor or epidermal growth factor plus basic fibroblast growth factor), serum-free culture medium, grow in suspension culture to form aggregates of cells known as “neurospheres.”

A subject selected neural cell exhibits one or more of the following characteristics: multipotent, self-renewing, engraftable, plastic, and migratory. A subject selected neural cell is capable of differentiating into a neuron.

The selected neural cells can be proliferated in suspension culture or in adherent culture. When the selected neural cells are proliferating as neurospheres, human nestin antibody may be used as a marker to identify undifferentiated cells. The proliferating cells show little glial fibrillary acidic protein (GFAP) staining and little β-tubulin staining.

As noted above, a subject selected neural cell is selected for an apoE4⁻ phenotype. Subject selected neural cells that are apoE4⁻ produce low or undetectable levels of apoE4, or are genotypically apoE4⁻. Methods for selecting for apoE4⁻ phenotype are discussed in more detail below.

In some embodiments, a subject selected apoE4⁻ neural cell is further selected for an apoE3⁺ phenotype. Where a subject selected neural cell is selected for an apoE3⁺ phenotype, in some embodiments, the selected neural cell will express higher levels of apoE3 mRNA and/or produce higher levels of apoE3 polypeptide than a non-selected cell. In some embodiments, the level of apoE3 polypeptide produced by a subject selected neural cell is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, or at least about 10-fold, or greater, more apoE3 polypeptide than an unselected, parent neural stem cell.

### Genetic Modification

In some embodiments, a subject selected neural cell is genetically modified (e.g., before selection) to produce a higher level of apoE3 polypeptide than a parent cell. In these embodiments, an exogenous nucleic acid comprising a nucleotide sequence encoding apoE3 is introduced into a parent neural cell, thereby genetically modifying the parent neural cell and generating a genetically modified neural cell, which is selected for increased production of apoE3 polypeptide. In other embodiments, a subject selected neural cell is genetically modified to reduce or eliminate apoE4 expression. In some embodiments, a neural cell is genetically modified both to produce a higher level of apoE3 than a parent cell and to reduce or eliminate apoE4 expression.

In some embodiments, genetic modification to increase apoE3 levels and/or to reduce or eliminate apoE4 expression is carried out before selection for an apoE4⁻ phenotype. Thus, in some embodiments, a method for generating a subject selected neural cell, or a subject population of selected neural cells, comprises: a) genetically modifying a neural cell (e.g., a parent neural cell) such that the genetically modified neural cell expresses a higher level of apoE3 than the parent neural cell and/or such that the genetically modified neural cell exhibits reduced apoE4 expression compared to the parent neural cell; and b) selecting the genetically modified neural cell(s) for an apoE4⁻ phenotype, as described above.

Genetic Modification for Overexpression of apoE3

In some embodiments, a neural cell is genetically modified with a nucleic acid comprising a nucleotide sequence encoding an apoE3 polypeptide. Nucleotide sequences encoding apoE3 polypeptide are known in the art. See, e.g., GenBank Accession Nos. NM_{—}000384, X04506, AH003569; and Ludwig et al. (1987) DNA 6:363.

In some embodiments, an apoE3-encoding nucleic acid is contained within an expression vector that provides for expression of the encoded apoE3 mRNA and production of the encoded apoE3 polypeptide in a neural cell. The expression vector will provide a transcriptional and translational initiation region, which may be inducible or constitutive, where the apoE3-coding region is operably linked to and under the transcriptional control of the transcriptional initiation region, and a transcriptional and translational termination region.

Any expression vector known in the art can be used to express the apoE3 nucleic acid. An expression vector will generally include a promoter and/or other transcription control elements which are active in the cell, and appropriate termination and polyadenylation signals. Expression vectors generally have convenient restriction sites located near the promoter sequence to provide for the insertion of nucleic acid sequences encoding heterologous proteins (e.g., apoE3). A selectable marker operative in the expression host may be present.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641 648, 1999; Ali et al., Hum Mol Genet. 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

In some embodiments, the apoE3-encoding nucleotide sequence is operably linked to a neuron-specific control element (e.g., a promoter, an enhancer). Neuron-specific promoters and other control elements (e.g., enhancers) are known in the art. Suitable neuron-specific control sequences include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; and a CMV enhancer/platelet-derived growth factor-β promoter (see, e.g., Liu et al. (2004) Gene Therapy 11:52-60).

The recombinant expression vector will in some embodiments include one or more selectable markers. In addition, the expression vectors will in many embodiments contain one or more selectable marker genes to provide a phenotypic trait for selection of transformed host cells such as dihydrofolate reductase or neomycin resistance for eukaryotic cell culture.

Genetic modification of a parent neural cell with an apoE3 nucleic acid, to generate a genetically modified neural cell, is performed using methods known in the art (see Maniatis et al., in Molecular Cloning: A Laboratory Manual, Cold Spring Harbor Laboratory, N.Y. (1982)). Exogenous DNA may be introduced into a parent neural cell by viral-mediated infection (retrovirus, modified herpes virus, herpes-viral, adenovirus, adeno-associated virus, and the like) or direct DNA transfection (lipofection, calcium phosphate transfection, DEAE-dextran, electroporation, and the like).

Genetic Modification to Reduce or Eliminate apoE4 Expression

In some embodiments, a subject selected neural cell is genetically modified (e.g., before selection for an apoE4⁻ phenotype) such that apoE4 expression in the selected neural cell is reduced or eliminated. For example, in some embodiments, a cell is derived from an individual who has an apoE4^{+/-} or apoE4^{+/+} genotype; and the cell is genetically modified to knock out apoE4 production in the cell. In some embodiments, reduction of apoE4 expression is achieved by genetically modifying a parent neural cell with an apoE4-specific siRNA, thereby generating a genetically modified neural cell, which is selected for decreased production of apoE4 polypeptide.

Methods for design and production of siRNAs to a desired target are known in the art, and their application to apoE4 genes for the purposes disclosed herein will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference.

Publicly available tools to facilitate design of siRNAs are available in the art. See, e.g., DEQOR: Design and Quality Control of RNAi (available on the internet at cluster-1.mpi-cbg.de/Deqor/deqor.html). See also, Henschel et al. Nucleic Acids Res. 2004 Jul. 1; 32(Web Server issue):W113-20. DEQOR is a web-based program which uses a scoring system based on state-of-the-art parameters for siRNA design to evaluate the inhibitory potency of siRNAs. DEQOR, therefore, can help to predict (i) regions in a gene that show high silencing capacity based on the base pair composition and (ii) siRNAs with high silencing potential for chemical synthesis. In addition, each siRNA arising from the input query is evaluated for possible cross-silencing activities by performing BLAST searches against the transcriptome or genome of a selected organism. DEQOR can therefore predict the probability that an mRNA fragment will cross-react with other genes in the cell and helps researchers to design experiments to test the specificity of siRNAs or chemically designed siRNAs.

Target sites suitable for design of siRNA for use in reducing apoE4 expression include nucleotide sequences encoding human apoE4. See, e.g., GenBank Accession No. NM_{—}000041, the sequence of which is provided herewith as SEQ ID NO: 1.

Non-limiting, exemplary target regions within SEQ ID NO: 1 include:

Target Region 1:

| | |
|---|---|
| 5′-ctgatggacg agaccatgaa-3′, | (SEQ ID NO:2) |
| corresponding to nucleotides 241-260 | |
| of the nucleotide sequence set forth | |
| in SEQ ID NO: 1. | |
| Target Region 2: | |
| 5′-cggctgggcg cggacatgga-3′, | (SEQ ID NO:3) |
| corresponding to nucleotides 361-380 | |
| of the nucleotide sequence set forth | |
| in SEQ ID NO: 1. | |
| Target Region 3: | |
| 5′-caggccgggg cccgcgaggg-3′, | (SEQ ID NO:4) |
| corresponding to nucleotides 541-560 | |
| of the nucleotide sequence set forth | |
| in SEQ ID NO: 1. | |

siNA molecules can be of any of a variety of forms. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. siNA can also be assembled from two separate oligonucleotides, where one strand is the sense strand and the other is the antisense strand, wherein the antisense and sense strands are self-complementary. In this embodiment, each strand generally comprises nucleotide sequence that is complementary to nucleotide sequence in the other strand; such as where the antisense strand and sense strand form a duplex or double stranded structure, for example wherein the double stranded region is about 15 base pairs to about 30 base pairs, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 nucleotides to about 25 or more nucleotides of the siNA molecule are complementary to the target nucleic acid or a portion thereof).

Alternatively, the siNA can be assembled from a single oligonucleotide, where the self-complementary sense and antisense regions of the siNA are linked by a nucleic acid-based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof.

The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate.

In certain embodiments, the siNA molecule contains separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In certain embodiments, the siNA molecules comprise nucleotide sequence that is complementary to nucleotide sequence of a target gene. In another embodiment, the siNA molecule interacts with nucleotide sequence of a target gene in a manner that causes inhibition of expression of the target gene.

As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. siNAs do not necessarily require the presence of nucleotides having a 2′-hydroxy group for mediating RNAi and as such, siNA molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.”

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence a target gene at both the post-transcriptional level or the pre-transcriptional level. In a non-limiting example, epigenetic regulation of gene expression by siNA molecules of the invention can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

siNA molecules contemplated herein can comprise a duplex forming oligonucleotide (DFO) see, e.g., WO 05/019453; and US 2005/0233329, which are incorporated herein by reference). siNA molecules also contemplated herein include multifunctional siNA, (see, e.g., WO 05/019453 and US 2004/0249178). The multifunctional siNA can comprise sequence targeting, for example, two regions of apoE4.

siNA molecules contemplated herein can comprise an asymmetric hairpin or asymmetric duplex. By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region.

Stability and/or half-life of siRNAs can be improved through chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein, describing various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; each of which are hereby incorporated in their totality by reference herein). In view of such teachings, similar modifications can be used as described herein to modify the siNA nucleic acid molecules of disclosed herein so long as the ability of siNA to promote RNAi is cells is not significantly inhibited.

Short interfering nucleic acid (siNA) molecules having chemical modifications that maintain or enhance activity are contemplated herein. Such a nucleic acid is also generally more resistant to nucleases than an unmodified nucleic acid. Accordingly, the in vitro and/or in vivo activity should not be significantly lowered. Nucleic acid molecules delivered exogenously are generally selected to be stable within cells at least for a period sufficient for transcription and/or translation of the target RNA to occur and to provide for modulation of production of the encoded mRNA and/or polypeptide so as to facilitate reduction of the level of the target gene product.

Production of RNA and DNA molecules can be accomplished synthetically and can provide for introduction of nucleotide modifications to provide for enhanced nuclease stability. (see, e.g., Wincott et al., 1995, Nucleic Acids Res. 23, 2677; Caruthers et al., 1992, Methods in Enzymology 211, 3-19, incorporated by reference herein. In one embodiment, nucleic acid molecules of the invention include one or more (e.g., about 1, 2, 3, 4, 5, 6, 7, 8, 9, 10, or more) G-clamp nucleotides, which are modified cytosine analogs which confer the ability to hydrogen bond both Watson-Crick and Hoogsteen faces of a complementary guanine within a duplex, and can provide for enhanced affinity and specificity to nucleic acid targets (see, e.g., Lin et al. 1998, J. Am. Chem. Soc., 120, 8531-8532). In another example, nucleic acid molecules can include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., Wengel et al., WO 00/66604 and WO 99/14226).

siNA molecules can be provided as conjugates and/or complexes, e.g., to facilitate delivery of siNA molecules into a cell. Exemplary conjugates and/or complexes includes those composed of an siNA and a small molecule, lipid, cholesterol, phospholipid, nucleoside, antibody, toxin, negatively charged polymer (e.g., protein, peptide, hormone, carbohydrate, polyethylene glycol, or polyamine). In general, the transporters described are designed to be used either individually or as part of a multi-component system, with or without degradable linkers. These compounds can improve delivery and/or localization of nucleic acid molecules into cells in the presence or absence of serum (see, e.g., U.S. Pat. No. 5,854,038). Conjugates of the molecules described herein can be attached to biologically active molecules via linkers that are biodegradable, such as biodegradable nucleic acid linker molecules.

### Selection

A subject selected neural cell, or selected neural cell population, is selected for an apoE4⁻ phenotype. In some embodiments, the source of the neural cell is an apoE4⁻ individual. As noted above, in some embodiments, the source of the neural cell is an apoE⁺ individual, and the neural cell is genetically modified to reduce or eliminate apoE4 expression.

A subject apoE4⁻ selected neural cell, or selected neural cell population, is in some embodiments further selected (e.g., sorted) for an apoE3⁺ phenotype. In some embodiments, the neural cells are selected for high expression of apoE3 (e.g., the cell has an apoE3^{hi} phenotype. Selection can be carried out using well-known methods, including, e.g., any of a variety of sorting methods, e.g., fluorescence activated cell sorting (FACS), negative selection methods, etc. The selected cells are separated from non-selected cells, generating a population of selected (“sorted”) cells. A selected cell population can be at least about 75%, at least about 80%, at least about 85%, at least about 90%, at least about 95%, at least about 98%, at least about 99%, or greater than 99%, apoE3^{hi} (and apoE4⁻), where the selected cell population is at least about 75%, at least about 80%, at least about 85%, at least about 90%, at least about 95%, at least about 98%, at least about 99%, or greater than 99%, apoE3 neural cells.

Cell sorting (separation) methods are well known in the art. Procedures for separation may include magnetic separation, using antibody-coated magnetic beads, affinity chromatography and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells (propidium iodide [PI], LDS). Any technique may be employed which is not unduly detrimental to the viability of the selected cells. Where the selection involves use of one or more antibodies, the antibodies can be conjugated with labels to allow for ease of separation of the particular cell type, e.g. magnetic beads; biotin, which binds with high affinity to avidin or streptavidin; fluorochromes, which can be used with a fluorescence activated cell sorter; haptens; and the like. Multi-color analyses may be employed with the FACS or in a combination of immunomagnetic separation and flow cytometry.

In other embodiments, a parent neural cell is contacted with an agent that is detectably labeled and that provides for detection of apoE3 mRNA or apoE3 cDNA in the cell. Alternatively, a parent neural cell is contacted with an agent that is detectably labeled and that provides for detection of apoE3 polypeptide in the cell. Cells that are apoE3⁺ are included in the selected neural cell population.

In some embodiments, a parent neural cell is genetically modified, as described above, to express higher level of apoE3 mRNA and/or produce a higher level of apoE3 polypeptide than a parent neural cell. In these embodiments, the genetically modified apoE3 over-expressing neural cell can be selected for high level of apoE3 mRNA and/or apoE3 polypeptide.

### Source of Neural Cells

Multipotent parent neural cells can be obtained from embryonic, post-natal, juvenile neural tissue, or adult neural tissue. Parent neural cells are in some embodiments derived from stem cells. In some embodiments, the stem cells are embryonic stem cells. In other embodiments, the stem cells are iPS cells. In other embodiments, the stem cell is an adult stem cell, e.g., a neural stem cell.

In some embodiments, the parent (e.g., un-selected) neural cells are human neural cells. The parent neural cells are multipotent neural cells, e.g., are capable of differentiating into neurons, astrocytes, or oligodendrocytes. In some embodiments, the parent neural cell is autologous in relation to the prospective recipient, e.g., the parent neural cells are obtained from the prospective recipient (in other words, the donor and the prospective recipient are the same individual). In other embodiments, the parent neural cell is allogeneic in relation to the prospective recipient, e.g., the parent neural cells are obtained from a donor of the same species as the prospective recipient, but the donor is not the prospective recipient (e.g., the prospective recipient is a human, and the donor is a human who is not the prospective recipient). In other embodiments, the parent neural cell is xenogeneic in relation to the prospective recipient, e.g., the parent neural cell is obtained from a donor of a species different from the species of the prospective recipient (e.g., the parent neural cell is porcine, and the prospective recipient is a human).

In some embodiments, a parent neural cell is derived from a stem cell. For example, in some embodiments, a parent neural cell is derived from an embryonic stem cell. In other embodiments, a parent neural cell is derived from an iPS cell. In still other embodiments, a parent neural cell is derived from an adult stem cell, e.g., a neural stem cell.

iPS cells are generated from somatic cells, including skin fibroblasts, using, e.g., known methods. iPS cells produce and express on their cell surface one or more of the following cell surface antigens: SSEA-3, SSEA-4, TRA-1-60, TRA-1-81, TRA-2-49/6E, and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX 1, FGF4, ESG1, DPPA2, DPPA4, and hTERT. In some embodiments, an iPS cell expresses Oct-3/4, Sox2, Nanog, GDF3, REX 1, FGF4, ESG1, DPPA2, DPPA4, and hTERT. iPS can be induced to differentiate into neural cells that express one or more of: βIII-tubulin, tyrosine hydroxylase, AADC, DAT, ChAT, LMX1B, and MAP2. Methods of generating iPS are known in the art, and any such method can be used to generate iPS. See, e.g., Takahashi and Yamanaka (2006) Cell 126:663-676; Yamanaka et. al. (2007) Nature 448:313-7; Wernig et. al. (2007) Nature 448:318-24; Maherali (2007) Cell Stem Cell 1:55-70.

iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and Klf4. In some embodiments, somatic cells are genetically modified with one or more expression constructs comprising nucleotide sequences encoding Oct-4, Sox2, Nanog, and LIN28.

iPS cells can be induced to differentiate into neural cells using any of a variety of published protocols (see, e.g., Muotri et al., 2005, Proc. Natl. Acad. Sci. USA. 102:18644; Takahashi et al, 2007, Cell 131:861). For example, in some embodiments, iPS cells are cultured on mitotically inactivated (mitomycin C-treated) mouse embryonic fibroblasts (Specialty Media, Lavellette, N.J.) in DMEM/F12 Glutamax (GIBCO), 20% knockout serum replacement (GIBCO), 0.1 nM nonessential amino acids (GIBCO), 0.1 nM 2-mercaptoethanol (GIBCO), and 4 ng/ml bFGF-2 (R & D Systems). iPS cell neuronal differentiation can be induced by coculturing the iPS cells with PA6 cells for 3-5 weeks under the following differentiation conditions: DMEM/F12 Glutamax (GIBCO), 10% knockout serum replacement (GIBCO), 0.1 nM nonessential amino acids (GIBCO), and 0.1 mM 2-mercaptoethanol (GIBCO). Alkaline phosphatase activity can be measured using the Vector Red Alkaline Phosphatase substrate kit I from Vector Laboratories. Neuronal differentiation can be monitored by immunostaining with various neuronal cell markers.

Any suitable tissue source can be used to obtain parent neural cells for use in preparing a subject selected neural cell population. Parent neural cells can generally be prepared from any fetal or adult tissue that contains neural stem cells or neural progenitor cells. Suitable tissues include, but are not limited to, hippocampus, septal nuclei, cortex, cerebellum, ventral mesencephalon and/or spinal cord.

Suitable sources of neural cells include the CNS, including the cerebral cortex, cerebellum, midbrain, brainstem, spinal cord and ventricular tissue; and areas of the peripheral nervous system (PNS) including the carotid body and the adrenal medulla. Exemplary areas include regions in the basal ganglia, e.g., the striatum which consists of the caudate and putamen, or various cell groups, such as the globus pallidus, the subthalamic nucleus, the nucleus basalis, or the substantia nigra pars compacta. In some embodiments, the neural tissue is obtained from ventricular tissue that is found lining CNS ventricles (e.g., lateral ventricles, third ventricle, fourth ventricle, central canal, cerebral aqueduct, etc.) and includes the subependyma. Suitable sources of neural cells also include cells from bone marrow, e.g., bone marrow derived stem cells, and the like.

Non-autologous human neural stem cells can be derived from fetal tissue following elective abortion, or from a post-natal, juvenile or adult organ donor. Autologous neural tissue can be obtained by biopsy, or from patients undergoing neurosurgery in which neural tissue is removed, for example, during epilepsy surgery, temporal lobectomies and hippocampalectomies. Neural stem cells have been isolated from a variety of adult CNS ventricular regions, including the frontal lobe, conus medullaris, thoracic spinal cord, brain stem, and hypothalamus, and proliferated in vitro using the methods detailed herein. In each of these cases, the neural stem cell exhibits self-maintenance and generates a large number of progeny which include neurons, astrocytes and oligodendrocytes.

### Culturing Neural Stem Cells

A parent neural stem cell, or a selected neural cell, is cultured in vitro in a suitable medium. The following are non-limiting examples of methods of obtaining neural stem cells, and culture conditions suitable for a parent neural stem cell and/or a selected neural cell.

### EXAMPLE 1

Tissue comprising neural stem cells is obtained. Fragments of the tissue are first dissociated using standard techniques to yield a single-cell suspension. The cells are then plated on a surface that does not substantially inhibit proliferation (i.e., the surface permits at least 20% doubling in a 24 hour period). Suitable surfaces include tissue culture plastic and surfaces treated with fibronectin. The cells are plated in a suitable medium (e.g., DMEM/F-12, with 10% fetal calf serum) at a density ranging from about 10⁶ to 10⁷, and at a density of about 5×10⁶ to about 5×10⁷ cells per 100 mm dish. This step of plating on a suitable surface provides for the proliferation of neural cells (e.g., human progenitor cells). Approximately 16-36 hours later, the medium is replaced with a suitable growth medium, for instance one which contains N2 supplements and fibroblast growth factor (FGF-2). For example, the growth medium can also contain epidermal growth factor (EGF), PDGF A/B and/or medium conditioned by immortalized adult rat hippocampal progenitor cells. For example, a suitable growth medium is DMEM/F-12 with 5 μg/mL insulin, 100 μg/mL transferrin, 20 nM progesterone, 30 nM sodium selenite, 100 .mu.M putrescine and 40 ng/mL human recombinant FGF-2, 40 ng/ML human recombinant EGF, 20 ng/mL human recombinant PDGF A/B and 50% conditioned medium.

### EXAMPLE 2

Tissue from a particular neural region is removed from the brain using a sterile procedure, and the cells are dissociated using any method known in the art including treatment with enzymes such as trypsin, collagenase and the like, or by using physical methods of dissociation such as with a blunt instrument. Dissociation of fetal cells can be carried out in tissue culture medium. An exemplary medium for dissociation of juvenile and adult cells is low Ca²⁺ artificial cerebral spinal fluid (aCSF). Regular aCSF contains 124 mM NaCl, 5 mM KCl, 1.3 mM MgCl₂, 2 mM CaCl₂, 26 mM NaHCO₃, and 10 mM D-glucose. Low Ca²⁺ aCSF contains the same ingredients except for MgCl₂ at a concentration of 3.2 mM and CaCl₂ at a concentration of 0.1 mM. Dissociated cells are centrifuged at low speed, between 200 and 2000 rpm, e.g., between 400 and 800 rpm, and then resuspended in culture medium. The neural cells can be cultured in suspension or on a fixed substrate. In some embodiments, the neural cells are cultured in suspension cultures. Cell suspensions are seeded in any receptacle capable of sustaining cells, particularly culture flasks, culture plates or roller bottles, e.g., in small culture flasks such as 25 cm² culture flasks. Cells cultured in suspension are resuspended at from about 5×10⁴ cells/ml to about 2×10⁵ cells/ml. Cells plated on a fixed substrate are plated at approximately 2-3×10³ cells/cm², or 2.5×10³ cells/cm².

### EXAMPLE 3

Dissociated neural cells are placed into any known culture medium capable of supporting cell growth, including HEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some embodiments, a defined, serum-free culture medium is used. An exemplary culture medium is a defined culture medium comprising a mixture of DMEM, F12, and a defined hormone and salt mixture. Another exemplary defined culture medium is a defined culture medium as described in WO 95/00632.

### EXAMPLE 4

Another suitable culture medium comprises cell viability and cell proliferation effective amounts of the following components: (a) a standard culture medium being serum-free (containing 0-0.49% serum) or serum-depleted (containing 0.5-5.0% serum), known as a “defined” culture medium, such as Iscove's modified Dulbecco's medium (“IMDM”), RPMI, DMEM, Fischer's, alpha medium, Leibovitz's, L-15, NCTC, F-10, F-12, MEM and McCoy's; (b) a suitable carbohydrate source, such as glucose; (c) a buffer such as MOPS, HEPES or Tris, e.g., HEPES; (d) a source of hormones including insulin, transferrin, progesterone, selenium, and putrescine; (e) one or more growth factors that stimulate proliferation of neural stem cells, such as EGF, bFGF, PDGF, NGF, and analogs, derivatives and/or combinations thereof, e.g., EGF and bFGF in combination; (f) LIF.

### EXAMPLE 5

Component Final Concentration 50/50 mix of DMEM/F-12; 0.5× to 2.0× glucose, e.g., 1× glucose; 0.2% to 1.0% w/v glutamine, e.g., 0.6% w/v glutamine; 0.1 nM-10 mM NaHCO₃, e.g., 2 nM NaHCO₃; 0.1 nM-10 nM HEPES, e.g., 3 mM HEPES; 0.1 nM-10 mM apo-human transferrin, e.g., 5 mM apo-human transferrin; 1 μg/ml-1000 μg/ml human insulin, e.g., 100 μg/ml human insulin; 1 μg/ml-100 μg/ml putrescine, e.g., 25 μg/ml putrescine; 1 μM-500 μM selenium, e.g., 60 μM selenium; 1 nM-100 nM progesterone, e.g., 30 nM progesterone; 1 nM-100 nM human EGF, e.g., 20 nM human EGF; 0.2 ng/ml-200 ng/ml human bFGF, e.g., 20 ng/ml human bFGF; 0.2 ng/ml-200 ng/ml LIF, e.g., 20 ng/ml human leukemia inhibitory factor (LIF); 0.1 ng/ml-500 ng/ml heparin, e.g., 10 ng/ml heparin; and 0.1 μg/ml-50 μg/ml, e.g., 2 μg/ml CO₂ e.g., 5% CO₂.

Conditions for culturing should be close to physiological conditions. The pH of the culture medium should be close to physiological pH, e.g., between pH 6 and pH8, e.g., between about pH 7 to pH 7.8, e.g., pH 7.4. Physiological temperatures range between about 30° C. and about 40° C. Cells are cultured at temperatures from about 32° C. to about 38° C., e.g., between about 35° C. and about 37° C.

In some embodiments, the neural stem cells are cultured in serum-free media containing epidermal growth factor (“EGF”) or an analog of EGF, such as amphiregulin or transforming growth factor alpha (“TGF-α”), as the mitogen for proliferation. See, e.g., WO 93/01275, WO 94/16718. Further, basic fibroblast growth factor (“bFGF”) can be used, either alone, or in combination with EGF, to enhance long term neural stem cell survival.

In some embodiments, bone marrow-derived stem cells are the source of neural cells. For example, CD34⁺ hematopoietic progenitor cells derived from human bone marrow, fetal bone marrow and liver, cord blood, or adult peripheral blood are selected for expression of a cell surface marker, e.g., AC133, 5E12, etc., where the cell surface marker is indicative of a central nervous system stem cell (CNS-SC) which can initiate neurospheres (NS-IC). Methods for selecting neural progenitor cells from CD34⁺ hematopoietic progenitor cells are known in the art. See, e.g., U.S. Pat. Nos. 6,467,794; and 7,037,719. CD34 is also known as gp 105-120. Monoclonal antibodies to CD34 are commercially available, and CD34 monoclonal antibodies have been used to quantitate and purify lymphohematopoietic stem/progenitor cells for research and for clinical bone marrow transplantation. Antibodies to AC133 can be obtained or prepared as discussed in U.S. Pat. No. 5,843,633.

In some embodiments, neural cells are isolated from hematopoietic progenitor cells (e.g., CD34⁺ cells) derived from human bone marrow, fetal bone marrow and liver, cord blood, or adult peripheral blood by a method involving: a) combining a population comprising neural cells or neural-derived cells containing a fraction of NS-ICs with monoclonal antibody AC133 or monoclonal antibody 5E12 or both; b) selecting the cells that bind to monoclonal antibody AC133 or to monoclonal antibody 5E12 or to both monoclonal antibody AC133 and to monoclonal antibody 5E12, such that the selected cells are enriched in the fraction of NS-ICs as compared with the population of neural cells; c) combining the enriched fraction obtained in step b) with a monoclonal antibody that binds to CD45 antigen or a monoclonal antibody that binds to CD34 antigen or both; d) selecting and eliminating CD45⁺, CD34⁺, or CD45⁺CD34⁺ cells, such that the remaining cells are further enriched in the fraction of NS-ICs as compared with the enriched fraction obtained in step b); e) introducing at least one cell from the enriched fraction obtained in step d) to a culture medium capable of supporting the growth of NS-IC; and f) proliferating the introduced cell in the culture medium. The culture medium is one that is capable of supporting the growth of NS-IC. For example, a suitable culture medium comprises a growth factor such as leukocyte inhibitory factor (LIF), epidermal growth factor (EGF), basic fibroblast growth factor (FGF-2) and combinations thereof. The culture medium may further include a neural survival factor (NSF). See, e.g., U.S. Pat. No. 7,037,719 for methods of isolating a neural cell from hematopoietic progenitor cells.

### Further Genetic Modifications

In some embodiments, e.g., where a parent neural stem cell is genetically modified to express higher levels of apoE3 than an unmodified parent neural stem cell, the genetically modified neural cell can be further genetically modified to produce one or more biologically active polypeptides. In other embodiments, e.g., where the parent neural stem cell is not genetically modified to express higher levels of apoE3 than an unmodified parent neural stem cell, the parent neural stem cell is genetically modified to produce one or more biologically active polypeptides. In some embodiments, a selected neural cell or a selected neural cell population is further genetically modified, e.g., the neural cell or neural cell population is further genetically modified after selection for an apoE4⁻ phenotype.

When the genetic modification is for the production of a biologically active polypeptide, the polypeptide is one that is useful for the treatment of a given CNS disorder. For example, in some embodiments, a neural cell is genetically modified to provide for secretion of one or more growth factors. As used herein, the term “growth factor product” refers to a protein, peptide, mitogen, or other molecule having a growth, proliferative, differentiative, or trophic effect. Suitable growth factors include, but are not limited to, nerve growth factor (NGF), brain derived neurotrophic factor (BDNF), a neurotrophin (NT-3, NT-4/NT-5), ciliary neurotrophic factor (CNTF), amphiregulin, fibroblast growth factor (e.g., FGF-1, FGF-2), epidermal growth factor (EGF), transforming growth factor-α (TGFα), TGFβ, platelet-derived growth factor (PDGF), an insulin-like growth factor (IGF), and an interleukin.

Cells can also be genetically modified to express a growth factor receptor (r) including, but not limited to, p75 low affinity NGFr, CNTFr, the trk family of neurotrophin receptors (trk, trkB, trkC), EGFr, FGFr, and amphiregulin receptors. Cells can be genetically modified to produce various neurotransmitters or their receptors such as serotonin, L-dopa, dopamine, norepinephrine, epinephrine, tachykinin, substance-P, endorphin, enkephalin, histamine, N-methyl D-aspartate, glycine, glutamate, gamma amino butyric acid (GABA), acetylcholine (ACh), and the like. Useful neurotransmitter-synthesizing genes include tyrosine hydroxylase, aromatic L-amino acid decarboxylase, dopamine β-hydroxylase, phenylethanolamine N-methyltransferase, DOPA decarboxylase, glutamate decarboxylase, tryptophan hydroxylase, choline acetyltransferase, and histidine decarboxylase. Nucleic acids that encode various neuropeptides, which may prove useful in the treatment of CNS disorders, include substance-P, neuropeptide-Y, enkephalin, vasopressin, vasoactive intestinal polypeptide, glucagon, bombesin, cholecystokinin, somatostatin, calcitonin gene-related peptide, and the like.

### Further Selection

Before or after selection for an apoE4⁻ phenotype (and, in some embodiments, further selection for an apoE3⁺ phenotype), a neural cell can be subjected to one or more selections, e.g., for expression of a cytoplasmic or cell surface marker. For example, a neural cell can be selected for differentiation into a neuron. Neurons can be identified using antibodies to neuron specific enolase (“NSE”), neurofilament, tau, β-tubulin, or other known neuronal markers. In some embodiments, a cell population is selected against differentiation into, or the presence of, an astrocyte. Astrocytes can be identified using antibodies to glial fibrillary acidic protein (“GFAP”), or other known astrocytic markers. In some embodiments, a cell population is selected against differentiation into, or the presence of, an oligodendrocyte. Oligodendrocytes can be identified using antibodies to galactocerebroside, O4, myelin basic protein (“MBP”) or other known oligodendrocytic markers.

In some embodiments, a neural cell is selected for a subset of neurons. For example, differentiated neural stem cell cultures can be selected to produce a neuronal population that is highly enriched in GABA-ergic neurons. Such GABA-ergic neuron enriched cell cultures are useful in the potential therapy of excitotoxic neurodegenerative disorders, such as Huntington's disease or epilepsy.

### Compositions Comprising a Selected Cell Population

The present invention provides compositions, including pharmaceutical compositions, comprising a subject selected neural cell population.

For administration to a mammalian host, a subject selected neural cell population can be formulated as a pharmaceutical composition. A pharmaceutical composition can be a sterile aqueous or non-aqueous solution, suspension or emulsion, which additionally comprises a physiologically acceptable carrier (i.e., a non-toxic material that does not interfere with the activity of the active ingredient). Any suitable carrier known to those of ordinary skill in the art may be employed in a subject pharmaceutical composition. Representative carriers include physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

In some embodiments, a subject selected neural cell population is encapsulated, according to known encapsulation technologies, including microencapsulation (see, e.g., U.S. Pat. Nos. 4,352,883; 4,353,888; and 5,084,350). Where the selected neural cells are encapsulated, in some embodiments the selected neural cells are encapsulated by macroencapsulation, as described in U.S. Pat. Nos. 5,284,761; 5,158,881; 4,976,859; 4,968,733; 5,800,828 and published PCT patent application WO 95/05452.

A unit dosage form of a subject selected neural cell population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells.

A subject selected neural cell population can be cryopreserved according to routine procedures. For example, cryopreservation can be carried out on from about one to ten million cells in “freeze” medium which can include a suitable proliferation medium, 10% BSA and 7.5% dimethylsulfoxide. Cells are centrifuged. Growth medium is aspirated and replaced with freeze medium. Cells are resuspended as spheres. Cells are slowly frozen, by, e.g., placing in a container at −80° C. Cells are thawed by swirling in a 37° C. bath, resuspended in fresh proliferation medium, and grown as described above.

### Treatment Methods

The present invention further provides methods of treating neurodegenerative disorders and demyelinating diseases, generally involving administering a subject selected neural cell population. A subject selected neural cell or neural cell population can be used for transplantation into a heterologous (allogeneic), autologous, or xenogeneic host (recipient).

A subject selected cell population will in some embodiments be transplanted into a patient according to conventional techniques, into the CNS, as described for example, in U.S. Pat. Nos. 5,082,670 and 5,618,531, or into any other suitable site in the body. In one embodiment, the selected cells are transplanted directly into the CNS. Parenchymal and intrathecal sites are also suitable. It will be appreciated that the exact location in the CNS will vary according to the disease state. Cells may be introduced by, for example, stereotaxic implantation or intracerebral grafting into the CNS of patients.

In some embodiments, a subject selected cell population is administered as a cell suspension. In other embodiments, a subject selected cell population is administered as neurospheres. In other embodiments, a subject selected cell population is administered in an encapsulated form. In other embodiments, a subject selected cell population is contained with a reservoir, and the reservoir is implanted into the individual.

Transplantation can be carried out bilaterally, or, in the case of a patient suffering from Parkinson's Disease, contralateral to the most affected side. Surgery is performed in a manner in which particular brain regions may be located, such as in relation to skull sutures, particularly with a stereotaxic guide. Cells are delivered throughout any affected neural area, in particular to the basal ganglia, e.g., to the caudate and putamen, the nucleus basalis or the substantia nigra. Cells are administered to the particular region using any method which maintains the integrity of surrounding areas of the brain, e.g., by injection cannula. Suitable approaches and methods may be found in Neural Grafting in the Mammalian CNS, Bjorklund and Stenevi, eds., (1985).

A single dose of a subject selected neural cell population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells. In some embodiments, multiple doses of a subject selected neural cell population are administered to an individual in need of such treatment. Doses can be administered at regular intervals (e.g., once a week, once a month, once every 6 weeks, once every 8 weeks, once every 6 months, etc.). Alternatively doses beyond an initial dose can be administered according to need, as determined by a medical professional, e.g., based on reappearance of symptoms associated with a neurodegenerative or demyelinating disorder, etc.

Functional integration of a subject selected neural cell population into the host neural tissue can be assessed by examining the effectiveness of subject selected neural cell population on restoring various functions, including but not limited to tests for endocrine, motor, cognitive and sensory functions. Motor tests which can be used include those which quantitate rotational movement away from the degenerated side of the brain, and those which quantitate slowness of movement, balance, coordination, akinesia or lack of movement, rigidity and tremors. Cognitive tests include various tests of ability to perform everyday tasks, as well as various memory tests, including maze performance.

A subject selected neural cell population is useful in the treatment of various neurodegenerative diseases and other disorders such as demyelinating diseases. In some embodiments, a subject selected neural cell population replaces diseased, damaged or lost tissue in the host. In other embodiments, subject selected neural cell population augments the function of an endogenous affected host tissue.

Disorders that are treatable using a subject method include, but are not limited to, epilepsy, ischemia, cerebellar ataxia; neurodegenerative diseases such as Huntington's disease, Parkinson's disease, amyotrophic lateral sclerosis, and Alzheimer's disease; demyelinating diseases, including disseminated perivenous encephalomyelitis, multiple sclerosis, neuromyelitis optica, concentric sclerosis, acute, disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g. stroke, head injury, cerebral palsy).

### Subjects Suitable for Treatment

Subjects suitable for treatment with a subject method include individuals who have been diagnosed as having a neurodegenerative disorder, a demyelinating disease, acute brain injury, spinal cord injury, or other disorder or condition that involves neuronal cell death or dysfunction. Subjects suitable for treatment with a subject method also include individuals who have been treated for a neurodegenerative disorder or a demyelinating disease, and who have either failed to respond to the treatment, or who initially responded to the treatment, but relapsed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Role of Apolipoprotein E in Neurogenesis

### Materials and Methods

Reagents and animals. Antibodies against NeuN and Nestin were from Chemicon (Temecula, Calif.). Antibody against S100-beta was from Abcam (Cambridge, Mass.). Antibody against Doublecortin (Dcx) was from Santa Cruz Biotech (Santa Cruz, Calif.). Antibody against GFAP was from Invitrogen (Carlsbad, Calif.). Antibody against beta-III-tubulin was from Promega (Madison, Wis.). Rat anti-BrdU was from Abcam, and mouse anti-BrdU was from Chemicon. Wildtype and apoE knockout mice were from the Jackson Laboratory (Bar Harbor, Me.). Human apoE3 or apoE4 knock-in mice were from Taconic (Hudson, N.Y.). All mice were weaned at 21 days of age, housed in a barrier facility at the Gladstone Animal Core with a 12-h light/12-h dark cycle, and fed a chow diet containing 4.5% fat (Ralston Purina).

Cell cultures. Mouse embryonic stem (ES) cells with targeted insertion of the EGFP cDNA into mouse apoE locus were grown on a layer of fibroblasts (feeder cells) that contain the neo and LIF genes. The neo gene confers resistance to G418 and the secreted LIF is necessary to keep the ES cells in their undifferentiated state. Both the ES and the feeder cells were cultured on plates coated with 0.1% gelatin. The ES cells were induced for neuronal differentiation in vitro for 2-25 days, and the differentiation status was analyzed by immunostaining with antibodies against various differentiation-related markers.

In some experiments, neurons differentiated from ES cells in vitro for 20-25 days were treated with C6-conditioned medium for 24 hours. Then, the expression of neuronal markers and EGFP was analyzed by immunofluorescent staining and confocal microscopy.

BrdU injection and collection of mouse brains. Female mice [apoE3 knock-in (apoE3-KI), apoE4-KI, apoE knockout (apoE-KO), wildtype, GFAP-apoE3, and GFAP-apoE4] at ages of 6-7 months (or 12-13 months) received two intraperitoneal injections of BrdU six hours apart, and the brains were collected 1, 3 day(s) or 4, 10 weeks after the second injection with PBS perfusion.

Immunostaining and quantification of neurogenesis and astrocytogenesis. After fixing in 3% PFA for 3 days, 40 μm coronal sections were cut continuously by vibratome. Brain sections through the whole hippocampus were collected in order. Every 8 section was immunostained with anti-BrdU and/or other cellular marker antibodies. The single- or double-immunostained cells on both sites of the hippocampus of all stained sections were counted. The total number of positive cells per hippocampus was calculated by number of positive cells from all stained sections multiplying 8 because every 8^{th} section had been stained. 1 or 3 days after BrdU injection, the numbers of newborn cells (BrdU positive) and differentiating cells [immature neurons (BrdU- and Dcx-double positive) and astrocytes (BrdU- and S100-beta-double positive)] were measured. 4 or 10 weeks after injection, the numbers of survived newborn cells (BrdU positive) and fully differentiated cells [mature neurons (BrdU- and NeuN-double positive) and astrocytes (BrdU- and S100-beta-double positive)] were measured.

### Results

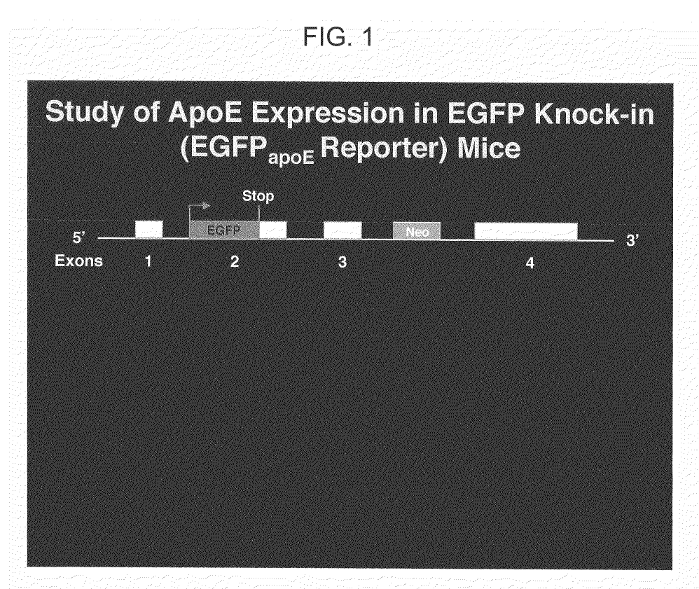

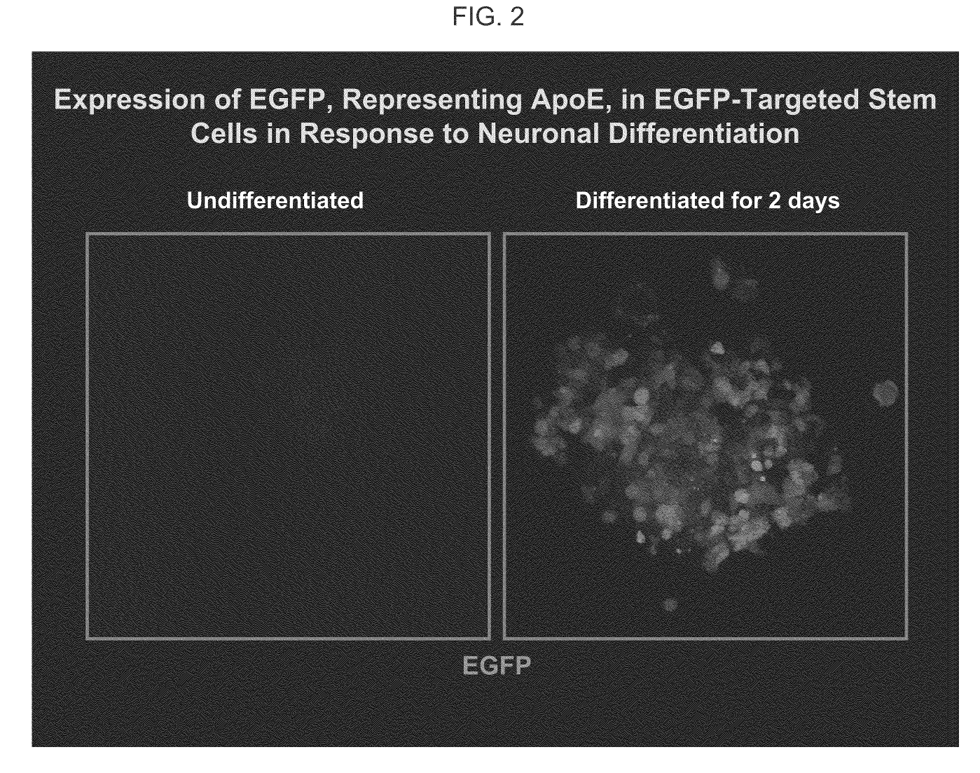

ApoE is expressed in ES cells during early neuronal differentiation. To study the regulation of apoE expression in various tissues and cells, mice were generated in which apoE expression can be detected with unprecedented sensitivity and resolution. cDNA encoding enhanced green fluorescent protein (EGFP) with a stop codon was inserted by gene targeting into the apoE gene locus (EGFP_{apoE}) immediately after the translation initiation site (FIG. 1). The EGFP_{apoE} reporter ES cells grown under an undifferentiation condition did not express EGFP that representing apoE (FIG. 2, left panel). However, induction of neuronal differentiation in vitro for 2 days turned on EGFP expression (FIG. 2, right panel), suggesting that apoE was expressed in ES cells during early neuronal differentiation.

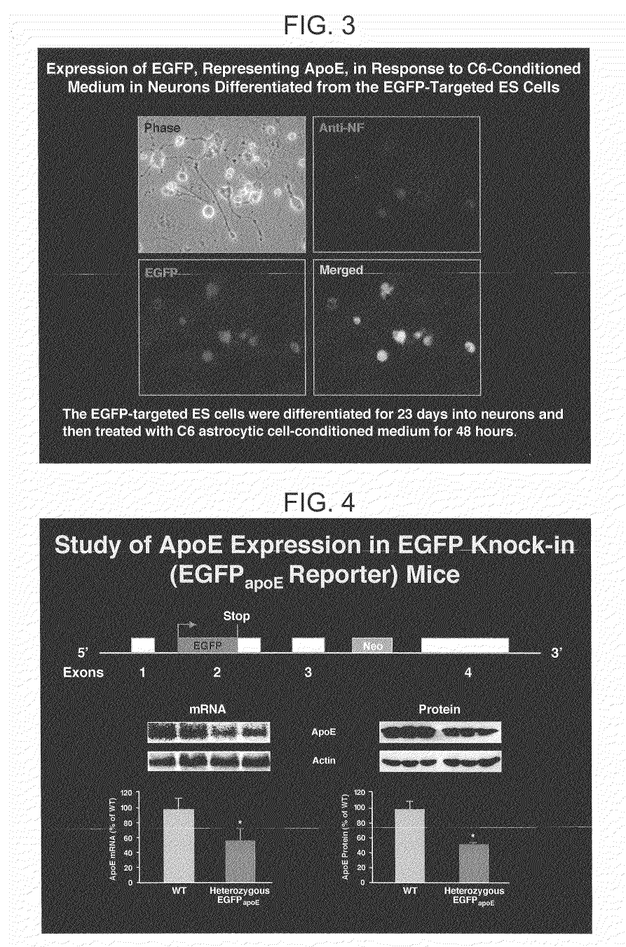

Astroglial regulation of apoE expression in ES cell-derived neurons. ES cells differentiated in vitro for 23 days were positive for neurofilament (a marker for mature neuron) staining (FIG. 3, top left and right panels), suggesting that they were mature neurons. Interestingly, these neurons shut off EGFP expression, suggesting that apoE was not expressed in mature neurons. However, treatment of these neurons with astrocyte C6-conditioned medium turned on EGFP expression (FIG. 3, lower left and right), suggesting that astrocyte-secreted factor(s) regulates neuronal expression of apoE.

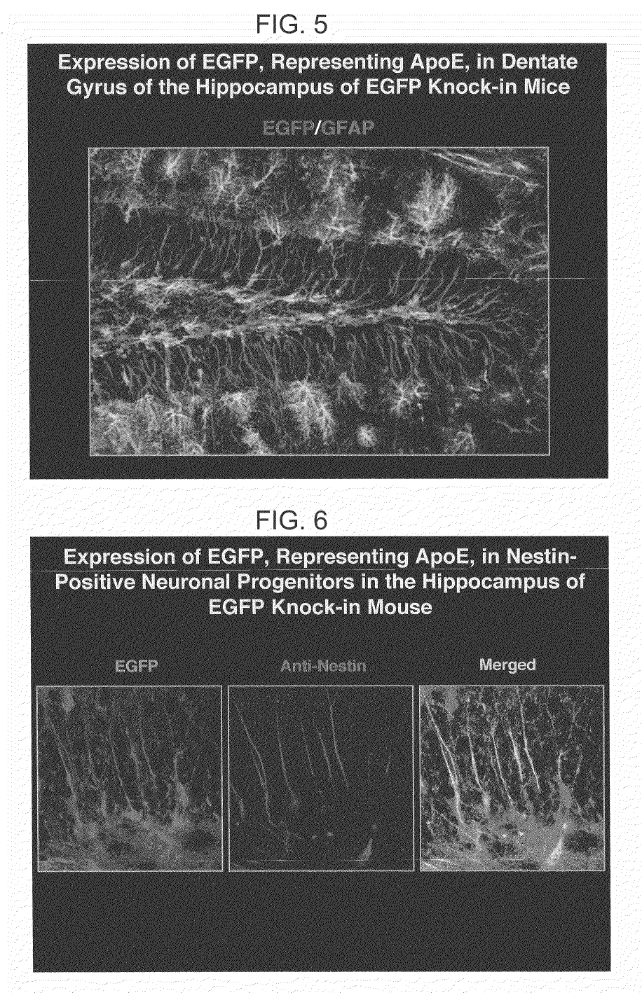

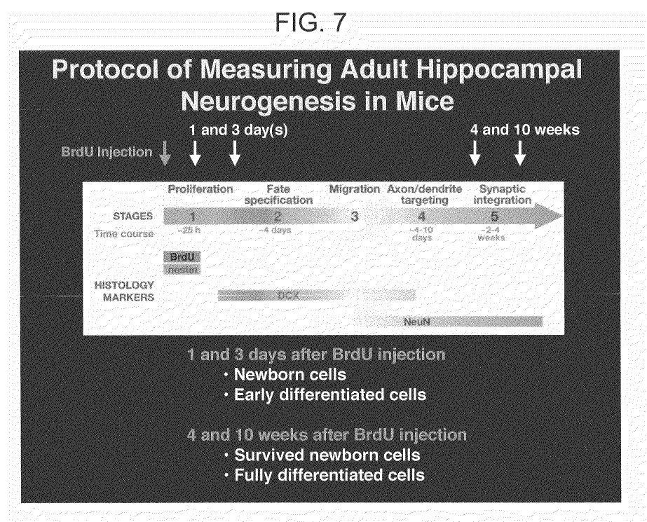

ApoE is expressed in neural progenitors in the hippocampus of mice. To study apoE expression in vivo in neural stem cells or progenitors, heterozygous EGFP_{apoE} reporter mice were used, in which one apoE allele was still active (FIG. 4). Confocal image revealed there were many EGFP-positive cells (representing apoE) along the subgranular zone (SGZ), which were negative for GFAP staining (FIG. 5). Based on the subgranular zone-location of these EGFP-positive cells, it was considered whether they were the neural progenitor cells. Immunostaining with an antibody against Nestin, a protein localized specifically in the processes of neural progenitor cells (FIG. 6, middle panel), revealed that the EGFP-positive cells in SGZ were also positive for Nestin. Thus, hippocampal neural progenitor cells express apoE, suggesting that apoE might play an important role in hippocampal neurogenesis.

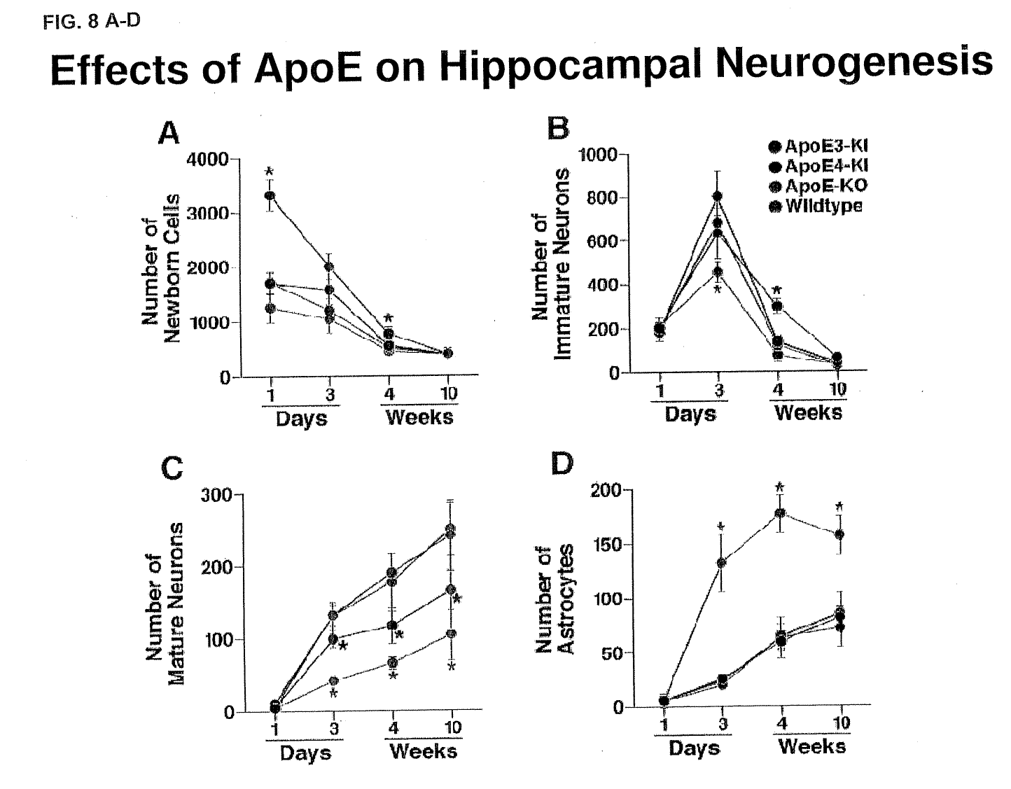

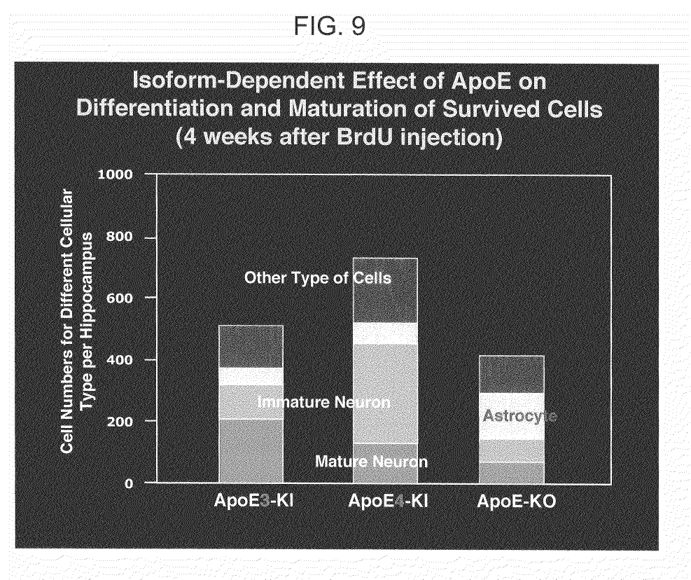

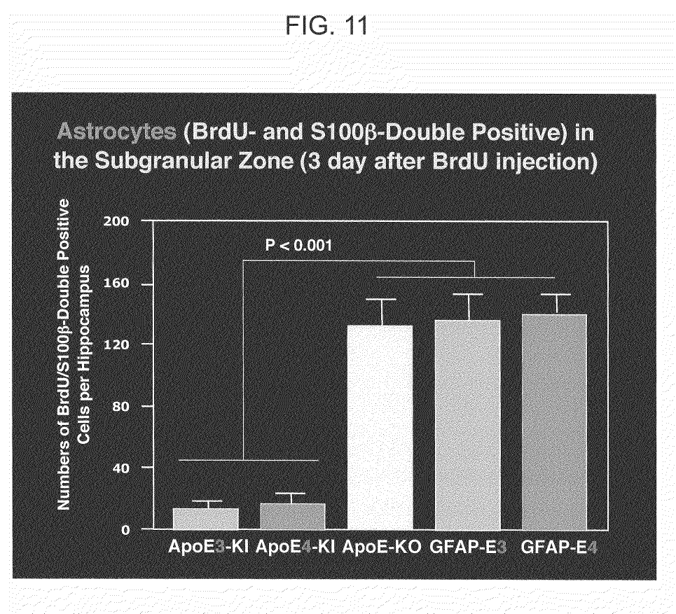

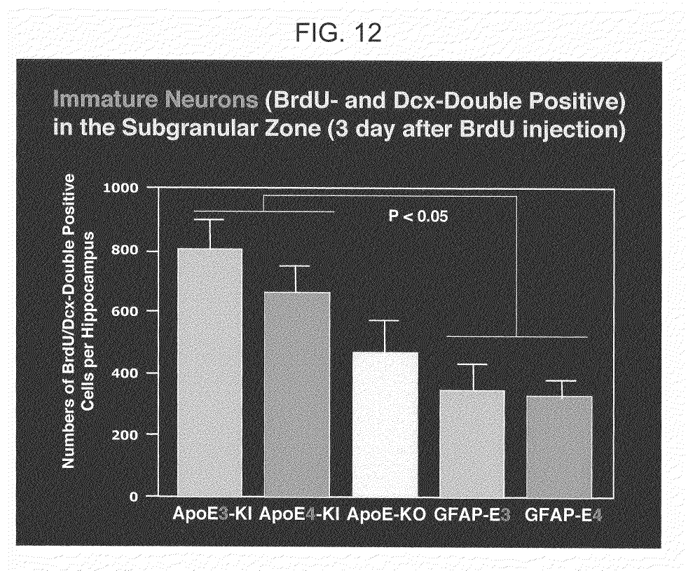

ApoE deficiency inhibits hippocampal neurogenesis but stimulates astrocytogenesis in mice. Analyses of hippocampal neurogenesis and astrocytogenesis by following the survival and differentiation of BrdU-positive cells in various transgenic mice at different time points after BrdU injection revealed that apoE knockout (apoE-KO) mice had much less newly generated mature neurons (BrdU- and NeuN-double positive), but much more newly generated astrocytes (BrdU- and S100-beta-double positive), than apoE3 knock-in (apoE3-KI) and apoE4 knock-in (apoE4-KI) mice at 3 days and 4 and 10 weeks after BrdU injection (FIGS. 8A-D and FIG. 9), suggesting that apoE deficiency inhibits hippocampal neurogenesis but stimulates astrocytogenesis. Thus, apoE might play an important role in neuronal fate determination. Interestingly, GFAP-apoE3 and GFAP-apoE4 transgenic mice, in which apoE was expressed only in astrocytes, had similar numbers of newly generated astrocytes (FIG. 11) and newly generated immature neurons (BrdU- and Dcx-double positive) (FIG. 12) to those seen in apoE-KO mice 3 days after BrdU injection, suggesting that astrocyte-derived apoE does not function in stimulating neuronal differentiation in the hippocampus.

ApoE4 inhibits neuronal maturation. Four weeks after BrdU injection, apoE4-KI mice had much more immature neurons, but much less mature neurons, than apoE3-KI mice (FIGS. 8A-D and FIG. 9), suggesting that apoE4 inhibits neuronal maturation. Ten weeks after BrdU injection, apoE4-KI mice still had significant less mature neurons, although immature neurons decreased to almost baseline in both mice (FIG. 8A-D). These results suggest that those immature neurons found in apoE4-KI mice at 4 weeks after BrdU injection died later.

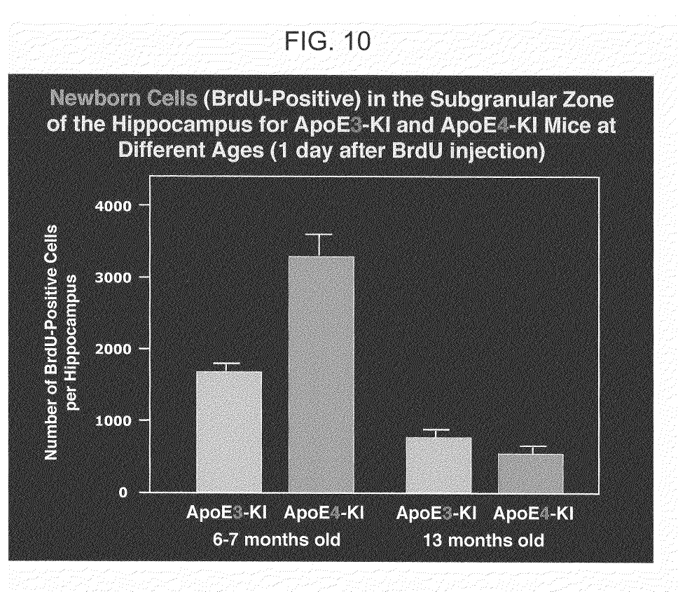

ApoE4 stimulates neural stem cell proliferation in young but not old mice. One day after BrdU injection, apoE4-KI mice had two-fold more BrdU-labeled cells in SVZ than apoE3-KI mice (FIG. 8A-D), suggesting that apoE4 stimulates neural stem cell proliferation. However, this stimulation only occurred in young, but not old, apoE4-KI mice (FIG. 10), suggesting an age-dependent decline of neural stem cell proliferation in response to apoE4.

ApoE4 stimulates BrdU-labeled newborn cell death. Although apoE4-KI mice had two-fold more BrdU-labeled cells than apoE3-KI mice one day after BrdU injection (FIG. 8A-D), 3 days after BrdU injection, apoE4-KI and apoE3-KI mice had similar numbers of BrdU-labeled cells (FIG. 8A-D), suggesting that many of the BrdU-labeled newborn cells died within 3 days in apoE4-KI mice. Thus, apoE4 stimulates BrdU-labeled newborn cell death during the early stage of neurogenesis.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A population of selected neural cells, wherein the neural cells are selected for having an apolipoprotein E4 negative (apoE4⁻) phenotype.

2. The population of claim 1, wherein the selected neural cells are genetically modified with an apolipoprotein E3 (apoE3) nucleic acid comprising a nucleotide sequence encoding apoE3, where the genetically modified neural cells produce a higher level of apoE3 protein than a control parent neural cell that is not genetically modified with the apoE3 nucleic acid.

3. The population of claim 1, wherein the selected neural cell is derived from a stem cell.

4. The population of claim 3, wherein the stem cell is an embryonic stem cell or an induced pluripotent stem cell.

5. The population of claim 3, wherein the stem cell is an adult stem cell.

6. The population of claim 5, wherein the adult stem cell is a neural stem cell.

7. The population of claim 3, wherein the stem cells differentiate into neurons.

8. The population of claim 1, wherein the selected neural cells are genetically modified to produce a neural growth factor, a neuroactive peptide, or a mitogen active on neural cells.

9. The population of claim 1, wherein the neural cells are further selected for a phenotype associated with a subset of neurons.

10. The population of claim 9, wherein the neural cells are selected for a GABAergic phenotype.

11. The population of claim 1, wherein the neural cells are derived from an individual having an apoE4⁺ genotype, and wherein the neural cells are genetically modified to reduce expression of apoE4.

12. A composition comprising the population of claim 1; and
a pharmaceutically acceptable excipient.

13. A method for treating a neurodegenerative disorder in a mammalian subject suffering from a neurodegenerative disorder, the method comprising administering to the mammalian subject an effective number of cells of the population of claim 1.

14. The method of claim 13, comprising administering from at about 10⁴ cells to about 10⁹ cells per dose.

15. The method of claim 13, comprising administering multiple doses of the cell population.

16. The method of claim 13, wherein the cell population is administered by injection at or near a site of central nervous system injury, damage, or lesion.

17. The method of claim 13, wherein the cell population is encapsulated.

18. The method of claim 13, wherein the disorder is Alzheimer's disease, Huntington's disease, Parkinson's disease, or amyotrophic lateral sclerosis.

19. The method of claim 13, wherein the disorder results from brain injury or spinal cord injury.

20. A method for treating a demyelinating disease in a mammalian subject suffering from a demyelinating disease, the method comprising administering to the mammalian subject an effective number of cells of the population of claim 1.

21. The method of claim 20, comprising administering from at about 10⁴ cells to about 10⁹ cells per dose.

22. The method of claim 20, comprising administering multiple doses of the cell population.

23. The method of claim 20, wherein the cell population is administered by injection at or near a site of central nervous system injury, damage, or lesion.

24. The method of claim 20, wherein the cell population is encapsulated.

25. The method of claim 20, wherein the demyelinating disease is multiple sclerosis.
